(19) United States
(12) Patent Application Publication
(10) Pub. No.: US 2018/0110531 A1
(43) Pub. Date: Apr. 26, 2018

(54) SYSTEM AND METHOD FOR HARVESTING BONE GRAFT

(71) Applicant: MIS IP Holding LLC, Denver, CO (US)

(72) Inventors: Brandon Arthurs, Wilmington, NC (US); Scott Noble, Denver, CO (US); Yuta Okkotsu, Aurora, CO (US)

(73) Assignee: MIS IP Holdings LLC, Denver, CO (US)

(21) Appl. No.: 15/790,253

(22) Filed: Oct. 23, 2017

Related U.S. Application Data
(60) Provisional application No. 62/411,639, filed on Oct. 23, 2016.

(51) Int. Cl. A61B 17/16; A61B 17/88; A61F 2/46

(52) U.S. Cl. CPC A61B 17/1635; A61B 17/3472; A61F 2/4644; A61B 17/8822

(57) ABSTRACT

Embodiments of the present disclosure are related to a minimally invasive bone graft harvesting system, apparatus, and method. Certain embodiments of include a sheath, a trocar, a trephine, and a plunger. A sheath and trocar can be locked together to function like a singular device, and is used to penetrate bone. A trephine is slideably placed through a sheath to bore through bone. A trephine further has interior surface features to help retain bone, and a longitudinal slit to facilitate ejecting bone. A plunger is placed through the trephine to eject bone.

### CROSS REFERENCE TO RELATED APPLICATIONS

This application claims benefit to U.S. Provisional Patent Application No. 62/411,639 entitled, “System and Method for Harvesting Bone Graft,” filed Oct. 23, 2016, the contents of which are incorporated by reference in its entirety for all purposes.

### FIELD OF THE INVENTION

Embodiments of the present disclosure are related to a bone graft harvesting system.

### BACKGROUND OF THE INVENTION

Many surgeries, including orthopedic, spinal, and neurosurgery use autologous or allogenic bone or bone graft substitutes to fuse bone. Autologous bone graft can be harvested from a number of locations, including the iliac crest, and has been considered the “gold standard” among graft materials. Autologous bone has the advantage of having osteogenic properties, shorter healing times, and favorable bone quality as compared to alternative materials.

However, harvesting autologous bone adds increased risk of morbidity due to the additional surgical steps. To reduce the risk of complications while harvesting autologous bone, there is a need for bone graft harvesting system, apparatus, and method that reduces the steps and instrumentation involved. There is a need to obtain bone graft using instruments that are intuitive to use while being effective. There is an overall need to more efficiently access a bone harvesting site and obtain autologous bone quickly, as to shorten the overall surgical time.

### SUMMARY OF THE INVENTION

Generally, embodiments of the invention are used for removal of bone from a body. Embodiments of the invention are directed to a system for harvesting or extracting autologous bone graft. Certain embodiments include a sheath, a trocar, a trephine, and a plunger to achieve bone removal in a minimally invasive manner.

In certain embodiments, a sheath has a duct, and is elongated about its longitudinal axis. A sheath allows slidable movement of other components therein. Certain embodiments of a sheath have a first end with a bevel. Embodiments of a bevel allow a first end of a sheath to be embedded in bone. Certain embodiments of a sheath have a second end with a handle.

In certain embodiments, a trocar has a first end with a tip, and a second end having a handle. In certain embodiments, a trocar is slideably placed in a sheath, and locked together. In certain embodiments, a handle of a sheath and a handle of a trocar can be held together as a single handling unit. An impact surface located on a handle is configured to be struck by a hammer. A trocar and sheath assembled as a single unit is used to pass through tissue to the bone. A trocar and sheath are impacted together with a hammer to further penetrate bone, until a portion of the sheath is embedded in bone.

In certain embodiments, a trephine includes a tube extending longitudinally. Certain embodiments of a trephine have a distal end with teeth for sawing into bone. Certain embodiments of a trephine have a proximal end having a handle. Holding a handle and rotating a trephine about its longitudinal axis allows boring into bone. A trephine distal end has an interior surface feature configured for retaining bone as the trephine is advanced into bone. Certain embodiments have a slit originating from the distal end of the trephine and extending towards a proximal end. Certain embodiments of a trephine have more than one slit. Certain embodiments of a trephine are slideably placed in a sheath, where the sheath is used to guide the trephine into the bone.

In certain embodiments, a plunger is configured to eject bone from a trephine. Certain embodiments of a plunger have an ejection end. Certain embodiments of a plunger have a handle end.

### DESCRIPTION OF THE DRAWINGS

FIG. 1: The bone harvesting system found in certain embodiments.

FIG. 2A: Top view of a sheath in certain embodiments.

FIG. 2B: Front view of a sheath in certain embodiments.

FIG. 2C: Side view of a sheath in certain embodiments.

FIG. 2D: Perspective view of a sheath in certain embodiments.

FIG. 3: Cross-sectional view of a sheath in certain embodiments.

FIG. 4A: Top view of a trocar in certain embodiments.

FIG. 4B: Front view of a trocar in certain embodiments.

FIG. 4C: Side view of a trocar in certain embodiments.

FIG. 4D: Perspective view of a trocar in certain embodiments.

FIG. 5: Perspective view of a trephine in certain embodiments.

FIG. 6A: Cross-sectional view of a trephine in certain embodiments.

FIG. 6B: Close-up view of a distal end of a trephine in certain embodiments.

FIG. 6C: Close up cross-sectional view of a trephine in certain embodiments

FIG. 7: Perspective view of a plunger in certain embodiments.

FIG. 8A: Insertion of a trocar in a sheath in certain embodiments.

FIG. 8B: Embodiment showing a trocar and sheath locked together.

FIG. 9A: Insertion of a trephine in a sheath in certain embodiments.

FIG. 9B: Insertion of a trephine in a sheath for extracting or harvesting bone, in certain embodiments.

FIG. 10A: Insertion of a plunger in a trocar in certain embodiments.

FIG. 10B: Insertion of a plunger in a trocar in certain embodiments.

FIG. 10C: Ejection of a bone graft from a trocar in certain embodiments.

### DETAILED DESCRIPTION

Examples directed towards a bone extraction system are described herein. It will be appreciated that the associated apparatuses or assemblies can be used in a number of clinical applications where extraction of bone is desired. Generally, the devices, systems, and methods for extracting bone described herein relate to a minimally invasive surgical method. The devices, systems, and methods described herein are not intended to limit the scope of the disclosure. It will be appreciated by those skilled in the art that the devices, systems, and methods can be used in various clinical applications.

It will be appreciated by those skilled in the art that the tools and components of such tools can be made of a number of medical grade materials. In certain embodiments, metals such as pure titanium, grade 5 titanium, titanium alloys, stainless steel alloys, and nitinol are used for certain features, though not limited to such metals or metal composites. In certain embodiments, materials such as polyphenylsulfone (Radel® R5500), polysulfone, acetal copolymers, polypropylenes, heat-stabilized polypropylenes, polyetheretherketone, polyaryletherketone, polyetherketone, and silicone are used for certain features, though not limited to such materials.

In general, embodiments of the invention are performed in conjunction with a number of instruments, including, but not limited to bone-imaging devices such as, for example, biplanar fluoroscopes (also referred to as C-Arm Fluoroscopes), and electrotransmitters, nerve monitoring systems, and related accessories. In general, the system, method, and apparatus related to certain embodiments are performed after disinfection, and other standard procedures and practices related to surgery and/or spinal surgeries known to persons having ordinary skill in the art.

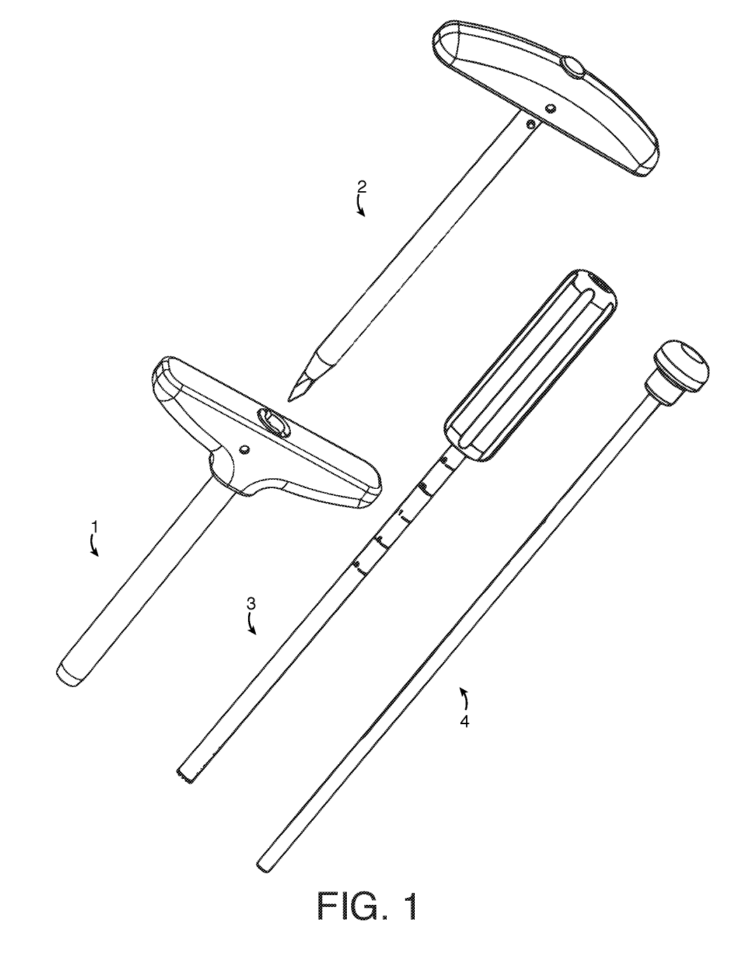

In some examples, the devices, systems, and methods of extracting bone include a number of instruments. Referring to FIG. 1, certain embodiments of a system include a sheath 1, a trocar 2, a trephine 3, and a plunger 4 to accomplish harvesting bone. It will be appreciated that the instruments required to accomplish bone extraction are not limited to such instruments. Certain embodiments of a bone extracting system include, for example, hammers, slap hammers, mallets, guide wires, Jamshidi needles, cannula, tissue retractors, rongeurs, scalpels, among others. It will be appreciated that certain instruments may be inserted into a body after an initial incision at the surgical site.

Generally, embodiments of a minimally invasive system, method, and apparatuses for bone extraction include a sheath, a trocar, a trephine, and a plunger. A sheath and trocar can be assembled and locked together to function like a singular device, where such assembly is used to penetrate bone. Once a sheath is embedded in bone, a trocar can be unlocked from the trephine and removed. A trephine is inserted through the sheath. A trephine is rotated and bored through bone. In certain situations, a rocking movement allows the bone to break off within one end of a trephine. A trephine is removed, and a plunger is used to eject a bone core from the trephine. In certain embodiments, a hammer is included with the system to facilitate placement of the instruments, or for removal of a bone core. It will be appreciated that bone core can be used in a number of surgical procedures, including, for example, spinal fusion.

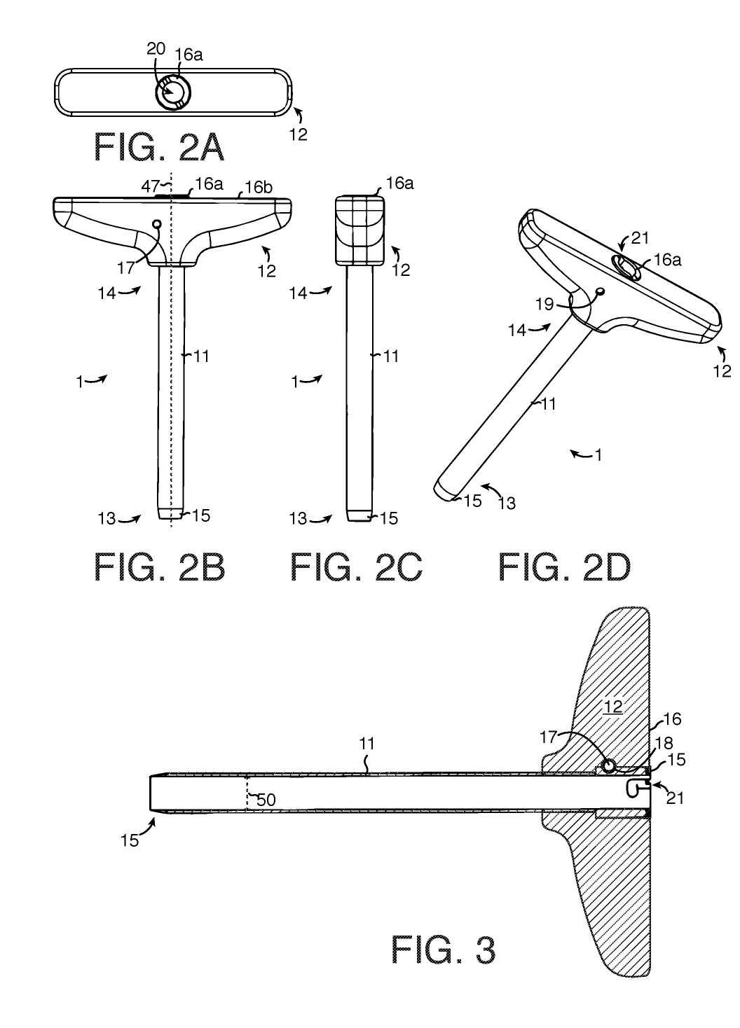

Referring to FIGS. 2A, 2B, 2C, and 2D, certain embodiments of a sheath 1 have a tube 11 elongated about its longitudinal axis 47. A sheath 1 first end 13 has a bevel 15. In certain embodiments, a bevel 15 is configured to assist in embedding a first end 13 of a sheath 1 into cortical bone of a bone extraction site.

A handle 12 is attached to a sheath 1 second end 14. As seen in FIG. 3, a tube 11 extends through a handle 12, where an end region 16a of a tube extends further past a handle surface 16b. Referring also to FIGS. 2A, 2B, 2C, and 2D, in certain embodiments, an end region 16a is an impact area for a hammer or similar tool. Striking the handle end region 16a helps to embed a sheath 1 into the bone. Referring to FIGS. 2D and 3, in certain embodiments, a tube 11 is secured to a handle 12 by a fixation pin 17 passing through a notch 18 on the tube 11 and further through openings 19 located on the handle 12. Referring to FIG. 2A, certain embodiments of a sheath 1 have a duct 20 allowing passage of other instruments. In certain embodiments, a trocar 2, trephine 3, and a plunger 4 can be passed through a sheath 1.

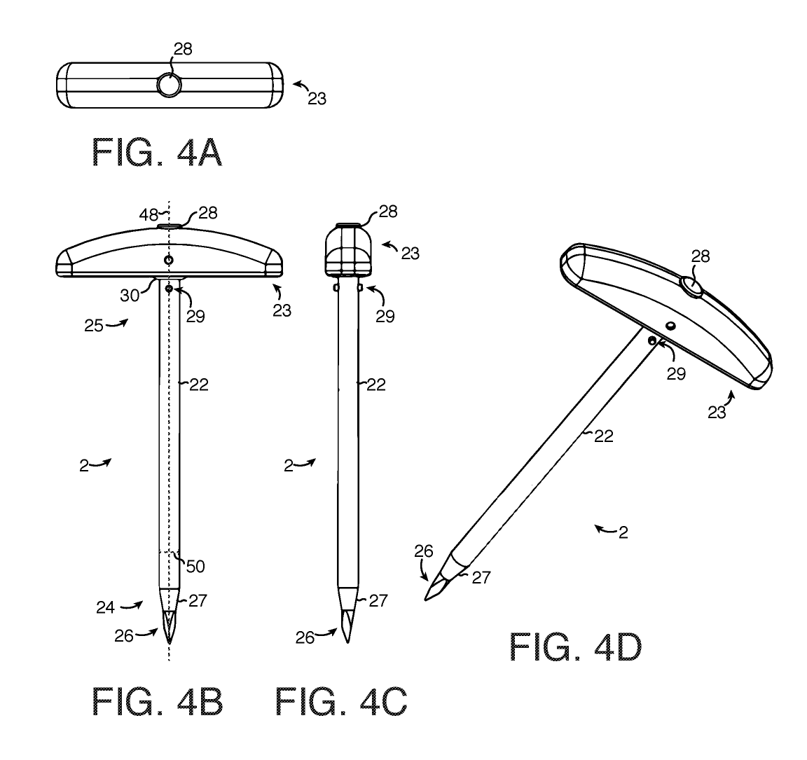

Referring to FIGS. 4A, 4B, 4C, and 4D, certain embodiments of a trocar 2 have a shaft 22 elongated about its longitudinal axis 48. A trocar 2 first end 24 has a tip 26. Certain embodiments of a tip are three sided, as shown, for example, in FIGS. 4B, 4C, and 4D. In certain embodiments, such tip has more than three sides, is conical, tapered, rounded, or is otherwise configured to allow tapping into bone. In certain embodiments, a tip is configured to penetrate bone. Certain embodiments of a trocar first end 24 have a taper 27.

Still referring to FIGS. 4A, 4B, 4C, and 4D, in certain embodiments, a trocar 2 second end 25 has a handle 23. In certain embodiments, a shaft 22 extends beyond a handle 23, and forms an upper impact surface 28. A hammer or a similar device can be used to strike an upper impact surface 28 as to tap and embed a trocar 2 through bone. In certain embodiments, a trocar 2 shaft 22 includes a bottom impact surface 30.

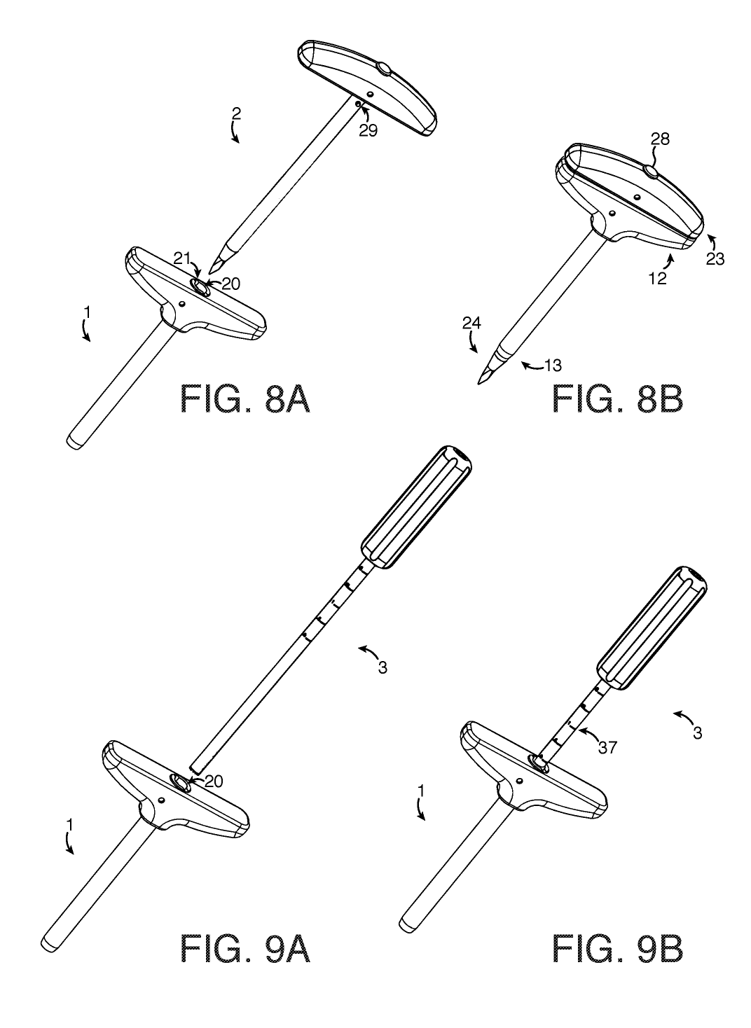

In certain embodiments, as shown in FIG. 8A, a trocar 2 is slideable through a sheath 1. A sheath has an inner diameter 50 (seen in FIG. 3) that is greater than a trocar shaft outer diameter 51 (seen in FIG. 4B). In certain embodiments, a pin 29 extends out of a shaft 22. Referring to FIGS. 2D, 3, 8A, and 8B, a sheath 1 tube 11 has a slot 21 configured to accept a trocar 2 pin 29. Once a trocar 2 is slid through a sheath 1 duct 20, the pin 29 follows the path of the slot 21, allowing the trocar 2 and sheath 1 to lock, as shown in FIG. 8B. Still referring to FIG. 8B, once locked, both a trocar 2 and sheath 1 can be held by the handle 12, 23. In certain embodiments, a sheath 1 handle 12 is a lower handle or a first handle, and a trocar 2 handle 23 is an upper handle or a second handle of a composite handle. Once a trocar 2 and sheath 1 are combined or assembled, a first end 24 of a trocar 2 protrudes beyond a sheath first end 13.

Referring to FIG. 4B, certain embodiments of a trocar 2 have a bottom impact surface 30 on a lower portion of a handle 23. In certain embodiments, a bottom impact surface 30 is a part of the shaft 22. In certain embodiments, when the trocar and sheath are assembled, a bottom impact surface 30 comes in contact and is in communication with the end region 16a of a sheath 2 (as seen for example in FIG. 2D). As seen in FIG. 8B, when an upper impact surface 28 of a trocar is struck, the force from the impact is transferred through the bottom impact surface 30, to the sheath end region 16a, and through the sheath. The impact force is also transferred through the trocar shaft 22. This configuration allows penetration of the first end 13 of a sheath 1 and the first end 24 of a trocar 2 through bone. In this manner, a trocar 2 and sheath 1 are struck together to pierce through bone. In certain embodiments, a trocar 2 and sheath 1 are struck until a portion of a sheath tube 11 or a sheath bevel 15 is embedded in the bone.

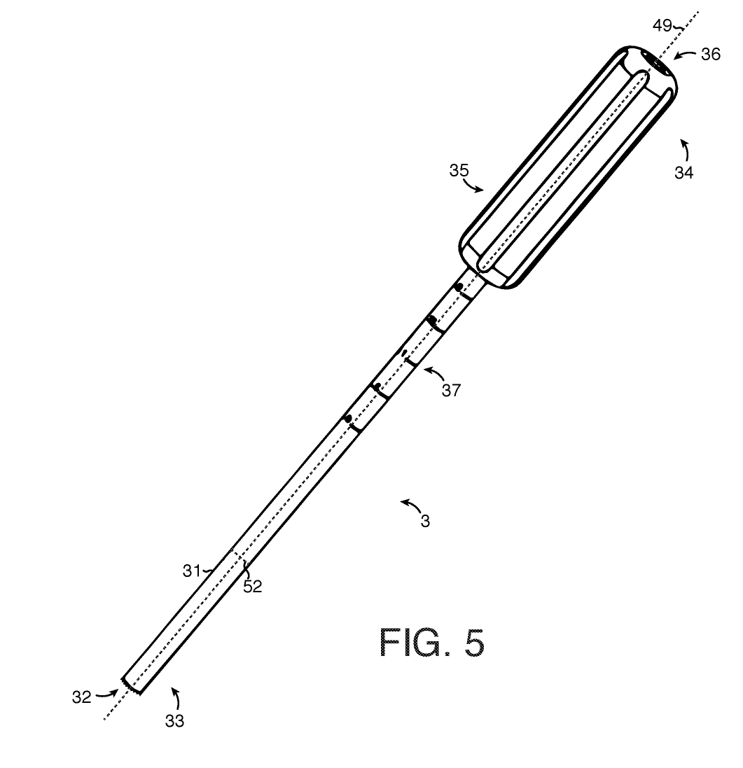

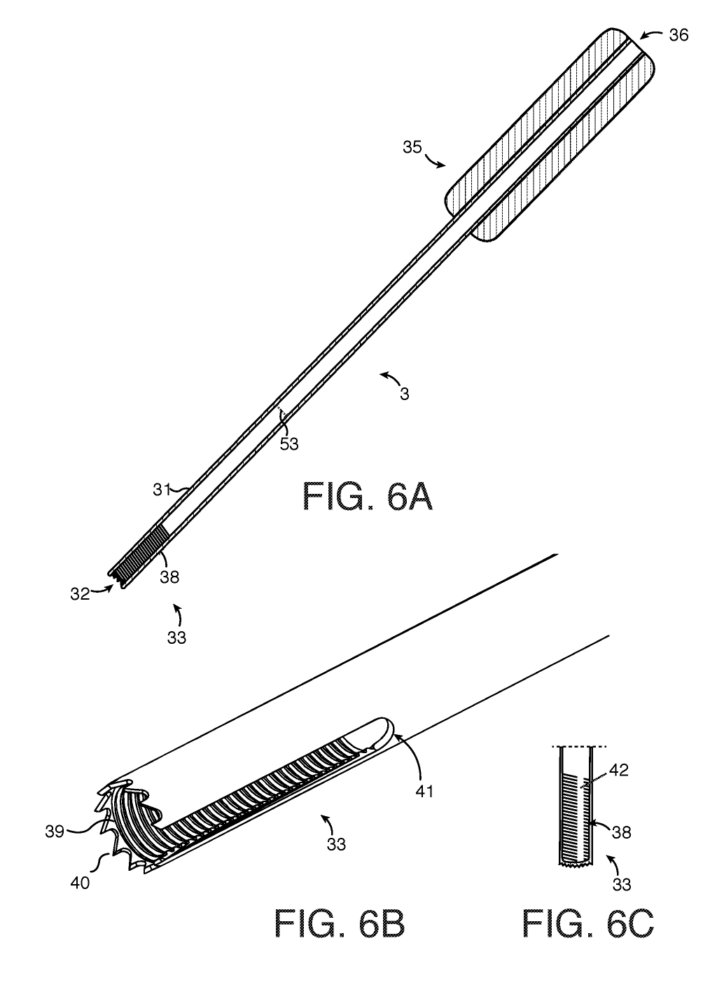

Referring to FIG. 5, embodiments of a trephine 3 have a shaft 31 with a distal end 33 and a proximal end 34. A trephine shaft distal end 33 further has a boring feature 32. Referring to FIG. 6B, certain embodiments of a boring feature include teeth 40, for example, a plurality of teeth. Referring to FIG. 5, a trephine shaft proximal end 34 has a handle 35. When in use, a distal end 33 having a boring feature 32 is butted against bone. While applying force against the bone, a piece of bone is bored out by holding the handle 35 and rotating the trephine 3 about its longitudinal axis 49. Referring to FIGS. 5 and 6, a trephine shaft 31 has a longitudinal opening 36. In certain embodiments, other tools, for example, a plunger 4 can be passed through an opening 36 and down a trephine shaft 31. In certain embodiments, a trephine shaft 31 has depth markers 37.

Referring to FIG. 6A showing a cross-sectional view of an embodiment of a trephine 3, a distal end 33 has an interior surface feature 38. An interior surface feature allows the trephine 3 to retain a bone core as it is advanced into bone. In certain embodiments, an interior surface feature includes threading 39 as shown, for example, in FIG. 6B. It will be appreciated by those skilled in the art that an interior surface feature can be a number of other features, for example, bulges, protrusions, clefts, and pits of varying shapes and sizes. In certain embodiments, a portion of the interior surface feature 38 is removed as to have a feature-less region, or a flute 42, as shown for example, in FIG. 6C. It will be appreciated that an interior surface feature may include one or more flutes in certain embodiments. In certain embodiments, a flute allows easier ejection of bone graft material.

Referring to FIG. 6B, in certain embodiments, a trephine distal end 33 has a longitudinal slit 41. A longitudinal slit 41 allows the trephine distal end 33 to expand while a bone core is being ejected, providing ejective relief. In certain embodiments one or more longitudinal slits are located at an end of a trephine 3. In certain embodiments, a longitudinal slit is in communication with the opening at a trephine distal end.

Referring to FIG. 9A, a trephine 3 is insertable into a sheath 1. In certain embodiments, a trephine 3 outer diameter 52 (seen in FIG. 5) is less than the sheath inner diameter 50 (seen in FIG. 3). In certain embodiments, once a sheath 1 and trocar 2 (seen in FIG. 8B) is embedded in bone, the trocar 2 is removed, leaving a sheath 1 in the bone. A trephine 3 is then inserted through the sheath duct 20, as to reach the bone harvesting site. Referring to FIG. 9B, the trephine 3 is used to bore graft material from the surgical site. Depth markers 37 are used to reference the depth, thus the approximate amount of bone that is being retained in the trephine as the trephine is advanced through the sheath 1.

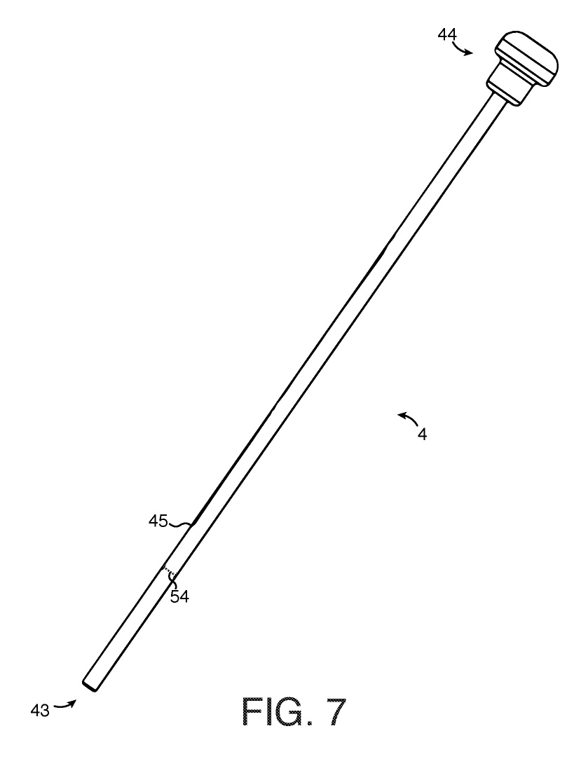

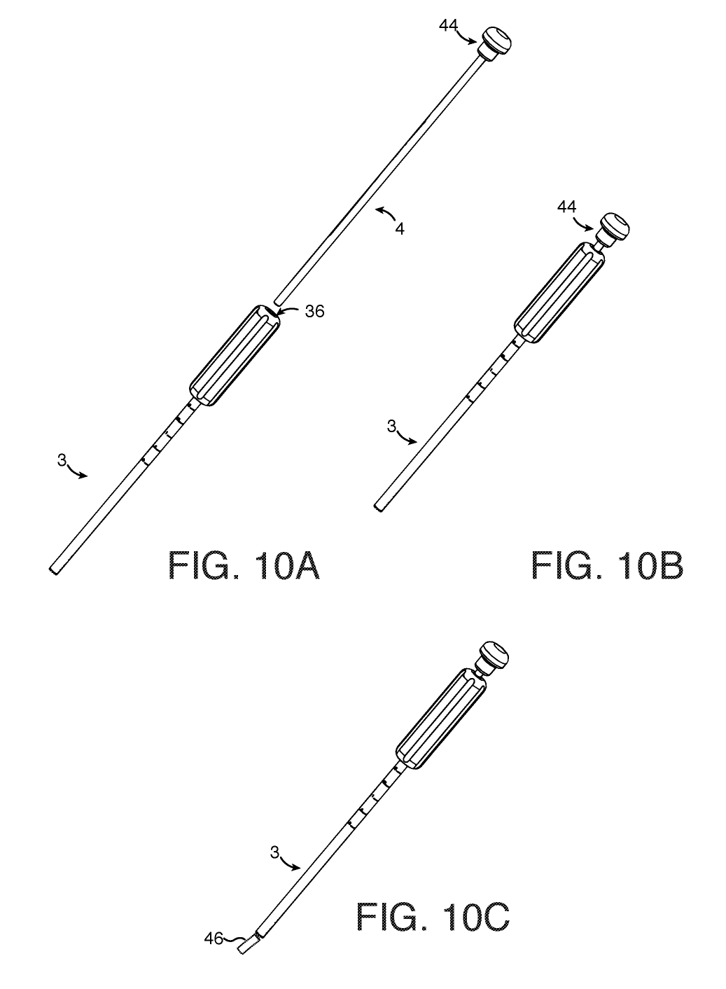

Referring to FIG. 7, a plunger 4 allows ejection of bone retained on a distal end 33 of a trephine 3. In certain embodiments, a plunger 4 has an elongated shaft 45 with an ejection end 43 and a handle end 44. Referring to FIGS. 10A, 10B, and 10C, a plunger 4 is inserted through a longitudinal opening 36 of a trephine 3. In certain embodiments, a plunger 4 elongated shaft 45 has an outer diameter 54 (seen in FIG. 7) that is less than the trephine 3 inner diameter 53 (seen in FIG. 6A). A plunger handle end 44 can be pushed to eject bone graft 46 from the trephine 3. In certain embodiments, a hammer can be used to strike a handle end 44 as to eject bone graft 46.

The illustrations of arrangements described herein are intended to provide a general understanding of the structure of various embodiments, and they are not intended to serve as a complete description of all the elements and features of apparatus and systems that might make use of the structures described herein. Many other arrangements will be apparent to those of skill in the art upon reviewing the above description. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The descriptive labels associated with the numerical references in the figures are intended to merely illustrate embodiments of the invention, and are in no way intended to limit the invention to the scope of the descriptive labels. The present systems, methods, means, and enablement are not limited to the particular systems, and methodologies described, as there can be multiple possible embodiments, which are not expressly illustrated in the present disclosures. It is also to be understood that the terminology used in the description is for the purpose of describing the particular versions or embodiments only, and is not intended to limit the scope of the present application.

Some embodiments, illustrating its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments are merely exemplary.

What is claimed is:

1. An extraction system for harvesting bone comprising:
a sheath, a trocar and a trephine;
the sheath having hollow profile with a longitudinal axis, a first end and a second end;
the first end of the sheath having a bevel on an external surface reducing toward the first end of the sheath, and the second end of the sheath comprising a lower handle;
the trocar comprising a shaft with a longitudinal axis, a first end, and a second end;
the first end of the shaft having a tip:
the second end of the shaft comprising an upper handle;
the shaft being slideably moveable within the hollow profile of the sheath along the longitudinal axis of the shaft;
said upper handle releasably lockable to said lower handle;
the trephine having a hollow profile with a longitudinal axis, a distal end, and a proximal end;
the trephine being slidably movable within the sheath;
the distal end of the trephine having an interior surface feature for retaining bone, and a longitudinal slit extending from the distal end toward the proximal end of the trephine;
the proximal end having a handle with hollow profile aligned with the hollow profile of the trephine.

2. The system in claim 1, wherein said surface feature comprises threading.

3. The system in claim 1, wherein said trephine distal end comprises a boring feature.

4. The system in claim 3, wherein said boring feature comprises a plurality of teeth.

5. The system in claim 1, wherein said upper handle and said lower handle is releasably lockable with a slot and pin.

6. The system in claim 1, further comprising a plunger having an ejection end and a handle end; and
said ejection end being slideably movable within the hollow profile of the trephine.

7. An extraction system for harvesting bone comprising:
a sheath, said sheath comprising a tube elongated about a longitudinal axis, said sheath further defining a duct connecting a first end and a second end, said second end of said sheath comprising a first handle;
a trocar, said trocar comprising a shaft elongated about a longitudinal axis, a first end of said trocar shaft having a tip,
the second end of said trocar shaft comprising a second handle,
said second handle having a locking connection with said first handle; and
a trephine, said trephine comprising a shaft elongated about a longitudinal axis, said trephine further defining a proximal end and a distal end, and an opening therebetween,
said distal end of the trephine shaft comprising a boring feature, an interior surface of said trephine shaft comprising an interior surface feature, and the proximal end of said trephine comprising a handle.

8. The system in claim 7, further comprising a plunger, said plunger comprising an ejection end and a handle end.

9. The system in claim 7, wherein said first end of said sheath comprises a bevel.

10. The system in claim 7, wherein said trephine distal end further comprises a longitudinal slit in communication with said distal end opening and extending from the distal end toward the proximal end.

11. The system in claim 7, wherein said interior surface feature further comprises a flute.

12. The system in claim 7, said sheath tube further comprising an end region extending past a handle surface; and said trocar second end further comprises an upper impact surface and a bottom impact surface; wherein said trocar bottom impact surface is in communication with said sheath end region when said trocar is locked with said sheath.

13. The system in claim 8, wherein said sheath has an inner diameter greater than an outer diameter of said trocar shaft and an outer diameter of said trephine; and wherein said trephine has an inner diameter greater than an outer diameter of said plunger.
